SEQUENCE LISTING (2 sequences)

SEQ ID NO: 1 - DNA, 29 bp, Artificial Sequence
  Laboratory-synthesized sequence
aatgatacggcgaccaccgagatctacac

SEQ ID NO: 2 - DNA, 24 bp, Artificial Sequence
  Laboratory-synthesized sequence
caagcagaagacggcatacgagat

(19) United States
(12) Patent Application Publication
(10) Pub. No.: US 2021/0371908 A1
(43) Pub. Date: Dec. 2, 2021

(54) COMPARING COPIES OF POLYNUCLEOTIDES WITH DIFFERENT FEATURES

(71) Applicant: ILLUMINA, INC., San Diego, CA (US)

(72) Inventors: Huihong You, San Diego, CA (US); YerPeng Tan, San Diego, CA (US)

(73) Assignee: ILLUMINA, INC., San Diego, CA (US)

(21) Appl. No.: 17/328,151

(22) Filed: May 24, 2021

Related U.S. Application Data
(60) Provisional application No. 63/031,230, filed on May 28, 2020.

(51) Int. Cl. C12Q 1/6816

(52) U.S. Cl. CPC C12Q 1/6816

(57) ABSTRACT

Provided is a method including making copies of two or more populations of polynucleotides including identifier sequences, wherein the copies are attached to a substrate, hybridizing oligonucleotides to the identifier sequences, and comparing an amount of oligonucleotides hybridized to the copies of the two or more populations of polynucleotides, wherein at least one feature differs between the two or more populations of polynucleotides or between the making of the copies of the two or more populations of polynucleotides attached to the substrate.

### CROSS-REFERENCE TO RELATED APPLICATIONS

This application claims benefit of priority from U.S. Provisional Patent Application No. 63/031,230, filed May 28, 2020, the entire contents of which is incorporated herein by reference.

### SEQUENCE LISTING

The instant application contains a Sequence Listing, created on May 18, 2021; the file, in ASCII format, is designated H2055887.txt and is 1 KB in size. The file is hereby incorporated by reference in its entirety into the instant application.

### BACKGROUND

Many current sequencing platforms use “sequencing by synthesis” (SBS) technology and fluorescence-based methods for detection. In some examples, numerous polynucleotides isolated from one or more populations of nucleotides to be sequenced are attached to a surface of a substrate and copied. SBS may then be performed on the surface-attached copies. Making copies of, or amplifying, polynucleotides, and sequencing the copies, increases a fluorescence signal emitted during sequencing and thereby enhances a sequencing process.

Copies of polynucleotides attached to a substrate may be synthesized by a method of solid-phase nucleic acid amplification, which allow amplification products to be immobilized on a solid support in order to form arrays including clusters of immobilized nucleic acid molecules. Each cluster or colony on such an array is a plurality of copies of a target polynucleotide strand and a plurality of immobilized polynucleotide strands complementary thereto. Cluster amplification methodologies, or clustering methods, are examples of methods wherein surface-attached copies of and complements to a target polynucleotide are synthesized for SBS. Some examples of suitable methodologies that can also be used to produce surface attached copies, etc., include bridge amplification, kinetic exclusion amplification (“ExAmp”), or others.

Clustering includes use of a polymerase to synthesize surface-attached clusters. However, a known issue with certain polymerases and polymerization methods is a quantitative synthesis bias related to various features of a target polynucleotide. For example, in some cases, a clustering method may be biased towards amplifying more copies of target polynucleotides that have a lower percentage of guanine (G)-cytosine (C) base pairs than polynucleotides that have a relatively higher GC content. In other cases, a clustering method may be biased towards amplifying more copies of relatively shorter target polynucleotides relative to relatively longer polynucleotides. In yet some other examples, other theoretical sources of bias may affect relative amplification levels of polynucleotides, such as polynucleotide sample preparation methods or other differences.

### SUMMARY

At least in view of the foregoing, sequencing techniques would therefore benefit from a method for determining existence of such biases in clustering and other amplification processes, and identifying, isolating, and modifying aspects of such techniques that may minimize such biases and result in more accurate sequencing results.

In an aspect, provided is a method, including making copies of two or more populations of polynucleotides including identifier sequences, wherein the copies are attached to a substrate, hybridizing oligonucleotides to the identifier sequences, and comparing an amount of oligonucleotides hybridized to the copies of the two or more populations of polynucleotides, wherein at least one feature differs between the two or more populations of polynucleotides or between the making of the copies of the two or more populations of polynucleotides attached to the substrate.

In an example, the at least one feature is selected from a length, a guanine-cytosine content, and a preparation method. In another example, the at least one feature includes a guanine-cytosine content. In still another example, the at least one feature includes a length. In yet another example, the at least one feature includes a preparation method. In a further example, at least one feature differs between the making of the copies of the two or more populations of polynucleotides attached to the substrate. In still a further example, the oligonucleotides include a fluorophore.

In an example, the method further includes detecting a difference between amounts of oligonucleotides hybridized to the copies of the two or more populations of polynucleotides attached to the substrate, wherein the difference is at least about 10%. In another example, the difference is at least about 20%. In still another example, the difference is at least about 30%.

In an example, the at least one feature includes a combination and the combination includes two or more of a guanine-cytosine content, a length, a preparation method, and the making of the copies of the two or more populations of polynucleotides attached to the substrate, the two or more populations of polynucleotides include three or more populations of polynucleotides, and the combination of each of the three or more populations of polynucleotides differs from the combination of another population of polynucleotides.

Another example further includes detecting a difference between amounts of oligonucleotides hybridized to the copies of two or more of the three or more populations of polynucleotides attached to the substrate, wherein the difference is at least about 10%. In an example, the difference is at least about 20%. In another example, the difference is at least about 30%.

In another aspect, provided is a method, including making copies of two or more populations of polynucleotides including identifier sequences, wherein the copies are attached to a substrate, hybridizing oligonucleotides including a fluorophore to the identifier sequences, and detecting an amount of oligonucleotides hybridized to the copies of the two or more populations of polynucleotides, wherein at least one feature differs between the two or more populations of polynucleotides or between the making of the copies of the two or more populations of polynucleotides attached to the substrate, and the at least one feature is selected from a length, a guanine-cytosine content, a preparation method, and the making of the copies of the two or more populations of polynucleotides attached to the substrate.

In an example, the at least one feature includes a guanine-cytosine content. In another example, the at least one feature includes a length. In still another example, the at least one feature includes a preparation method. In yet another example, at least one feature differs between the making of the copies of the two or more populations of polynucleotides attached to the substrate.

Another example further includes detecting a difference between an amount of oligonucleotides hybridized to copies of the two or more populations of polynucleotides, wherein the difference is at least about 10%. In an example, the difference is at least about 20%. In still another example, the difference is at least about 30%.

### BRIEF DESCRIPTION OF THE DRAWINGS

These and other features, aspects, and advantages of the present disclosure will become better understood when the following detailed description is read with reference to the accompanying drawings, wherein:

FIG. 1 shows a flow diagram in accordance with aspects of one example of method as disclosed herein.

FIG. 2 shows an illustration of elements of one example of a method in accordance with aspects of the present disclosure.

FIG. 3 is a graph showing, in one example, differences in mean intensity detected from fluorescently labeled oligonucleotides hybridized to copies of polynucleotides starting from different proportions of loaded DNA in accordance with aspects of the present disclosure.

FIG. 4 is a graph comparing, in one example, intensities of fluorescence detection following clustering of polynucleotides loaded at 40%, 50%, or 60% of total DNA in a clustering procedure.

FIG. 5 shows a flow diagram in accordance with aspects of one example of method as disclosed herein.

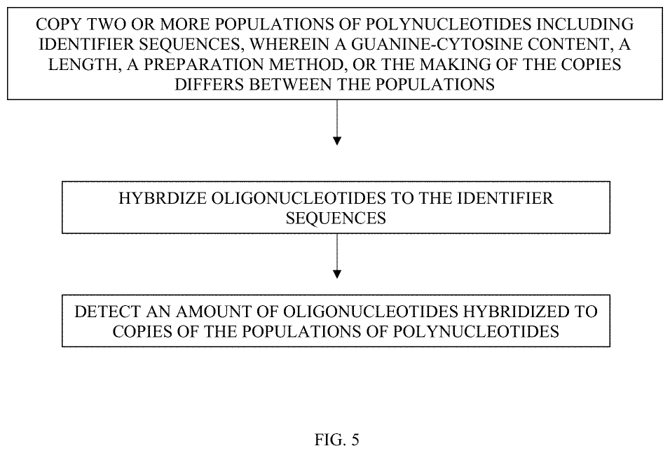

### DETAILED DESCRIPTION

This disclosure relates to a method for assessing bias in copying polynucleotides, such as part of an SBS process. In particular, included is a process for identifying presence of bias for making relatively more or fewer copies of polynucleotides from a given population relative to those from a different population. Polynucleotides of different populations may be distinguished from each other by features that differ from one population to the other. A feature may be any characteristic of polynucleotides of a population including physical attributes of the polynucleotide strands or processes to which the population of polynucleotides are subjected to as aspects of sample preparation.

For example, polynucleotides from one population may have a lower or higher ratio of C and/or G bases to A and/or T bases relative to polynucleotides from another population. In another example, polynucleotides from a population may be a number of nucleotides in length, with the length of polynucleotides of one population differing from the length of polynucleotides of another population. In another example, different populations of polynucleotides may have been subjected to differing preparation methods. For example, they may have been subjected to different methods of fragmentation of target molecules into shorter polynucleotides for copying and sequencing, or different methods of adding oligonucleotide sequences or identifiers to polynucleotides (a process sometimes referred to as indexing, index tagging, or barcoding, which is a way of tagging or indexing polynucleotides for identification of copies subsequently made thereof), or different methods of separating polynucleotide sequences from an initial sample (such as isolating selecting polynucleotides of a predetermined size or within a predetermined size range). In other examples, a method of cluster formation from one population of polynucleotides may differ from a method of cluster formation from a different population of polynucleotides.

In some examples, any feature, whether pertaining directly to physical characteristics of polynucleotides of different populations, or indirectly indicative of characteristics of polynucleotides themselves as a result of their preparation, storage, treatment, handling, or preparation, or clustering process, or related to other characteristics such as other components that may be present with the polynucleotides, etc., may differentiate two or more populations. A method as disclosed herein may be used to determine whether a difference in features results in a bias in copying, such as during a clustering process, that results to a disproportionately higher amount or rate of copying of polynucleotides from one population relative to another.

In some examples, populations may differ in regard to more than one feature (including from among GC content, length, preparation method, or process of making of copies such as during clustering). For example, populations may differ as to length (e.g., number of nucleotides in polynucleotides of a population) and as to GC content (e.g., in a relative amount of G and/or C residues in the population of polynucleotides compared to A and/or T residues in the population of polynucleotides). Or they may differ as to either of these and preparation method, of copying method during clustering, or any combination of two or more of the foregoing. In some examples, populations may differ as to one or more feature, or as to combinations of any two or more features, or as to combinations of any three or more features (such as length, GC content, or a method by which polynucleotides are prepared for copying or clustering, and or method of copying such as clustering method).

Differences in a method of preparation of different populations of polynucleotides, constituting features of the populations, may impart different structural characteristics on the populations, such as differences in effectiveness of obtaining polynucleotides of an intended size, consistency of polynucleotide size within a population, how many polynucleotides within a population correctly had adapter or other sequences attached thereto, etc., all of which could result in biases or copying differences that become evidence following clustering. The method disclosed herein can be used to ascertain such effects of differences in preparation method.

In some examples, a feature of one or more of the two or more populations of polynucleotides, including any of the foregoing features, or any two or more of the foregoing features in combination with each other, may be preselected. For example, it may be advantageous to determine whether a clustering process or other aspect of a copying process causes, increases, decreases, eliminated, or otherwise affects a bias for a polynucleotide length, GC content, preparation process, or other feature, or method of making copies, or any combination of two or more thereof. Thus, features of populations of polynucleotides may be preselected and configured to reflect such potential or hypothesized cause or source of bias, and the clustering or other copying process performed and amount of copies of the two or more populations of polynucleotides compared. A greater amount of copies of one population than another, normalized by starting amounts of each at the commencement of copying, may signify a bias for or against polynucleotides bearing the pre-selected feature under the copying conditions used.

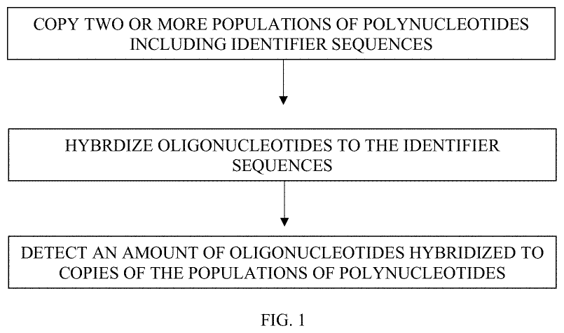

An example of such a method is illustrated in the flow diagram of FIG. 1. Two or more populations of polynucleotides are prepared for copying, such as by a clustering process. Included in the preparation process is addition to an oligonucleotide sequence to polynucleotides of a population, and another oligonucleotide sequence to polynucleotides of another population. In an example wherein more than two populations of polynucleotides are used, an oligonucleotide sequence may be added to polynucleotides of a population that differs from an oligonucleotide sequence added to polynucleotides of each of the other populations, such that polynucleotides of each population include an oligonucleotide sequence specific for the polynucleotides of that population and which differs from the oligonucleotides added to polynucleotides of any other population. Differences in sequences of such oligonucleotides, referred to as identifier sequences, may be such that they hybridize to an oligonucleotide having a sequence complementary thereto. For example, each identifier sequence added to polynucleotides of two or more populations thereof may be hybridizable to an oligonucleotide sequence to which identifier sequences of polynucleotides from any other of the two or more populations thereof are not hybridizable to. As further explained below, presence of sequence identifiers thereby differentiable between polynucleotides from different populations may permit identification of copies of polynucleotides from a given population as opposed to of any other population, in accordance with a method as disclosed herein.

Single-stranded polynucleotides of the two or more populations may then be copied, with copies attached to a substrate. For an example, copying may be performed, as mentioned above, according to a solid-state exclusion amplification clustering process, bridge amplification clustering, or other process. In a non-limiting example, 3-prime ends of polynucleotides may be hybridized to primer sequences attached to a substrate, and a polymerization process performed to create a complement to the polynucleotides, beginning with the surface-attached primer and extending to a complement to the 5-prime end of each polynucleotide. Polynucleotides from the two or more populations may then be amplified from the surface-attached complements thereof. According to a bridge-PCR process, as non-limiting example, free, 3-prime ends of the surface-attached complements to the polynucleotides of the two or more populations may then hybridize to another primer sequence attached to the substrate. The complements may then be copied by a polymerase reaction, resulting in copies of the polynucleotides of the two or more populations of polynucleotides, as well as complements thereto, extending from the surface. The surface-bound complements and copies may then be dehybridized from each other, and another polymerization round performed wherein the surface-attached copies of the polynucleotides of the two or more populations of polynucleotides, and complements thereto, are copied (following hybridization of their free 3-prime ends to surface-attached primers as initiation sites for a polymerase reaction), then complementary pairs of surface-attached polynucleotides dehybridized from each other. By repeating this process, clusters of copies of the polynucleotides of the two or more populations, and complements thereto, may be formed, attached to the substrate. Other, comparable methods of making copies of populations of polynucleotides may also be employed in other examples, whether PCR, rolling-circle amplification, multiple displacement amplification, random prime amplification, isothermal amplification, etc.

An amount of substrate-attached copies of polynucleotides from one of the two or more populations of polynucleotides may then be determined. For example, an oligonucleotide hybridizable to the identifier sequence of polynucleotides of one of the two or more populations may be added such that it hybridizes to said identifier sequence as present on said polynucleotides. The hybridizable oligonucleotide may include a detectable marker, such as a fluorescent marker capable of emitting detectable fluorescence upon stimulation by a given wavelength of electromagnetic radiation. By inducing such hybridized oligonucleotides to fluoresce, and detecting an amount of fluorescence emitted, an amount of copies of polynucleotides from one of the two or more populations can be assessed.

Said oligonucleotide can then be dehybridized, followed by incubation with another oligonucleotide, which other oligonucleotide is hybridizable to an identifier sequence of polynucleotides of another of the two or more populations of polynucleotides. Said other hybridizable oligonucleotide may include a detectable marker, such as a fluorescent marker capable of emitting detectable fluorescence upon stimulation by a given wavelength of electromagnetic radiation. By inducing such other hybridized oligonucleotides to fluoresce, and detecting an amount of fluorescence emitted, an amount of copies of polynucleotides from the other of the two or more populations can be assessed. In an example where potential copying bias caused or resulting from features, or combinations of features, of polynucleotides from more than two populations of polynucleotides, a process of hybridizing an oligonucleotide hybridizable to each identifier sequence of polynucleotides of individual populations of nucleotides, measuring an amount of hybridized oligonucleotide, followed by dehybridization thereof (if hybridization of another oligonucleotide is to follow), may be repeated to obtain a measurement of an amount of each type of oligonucleotide, as a measure of an amount of copies of polynucleotides from each of the two or more populations of polynucleotides.

In an example, a difference between an amount of oligonucleotides hybridized to copies of each of the two or more populations of polynucleotides may be detected by comparing relevant amounts of, for example, fluorescence emitted from oligonucleotides hybridizable to respective identifier sequences. For example, a sample may include two populations of polynucleotides including different identifier sequences and characterized by having different features. Different samples may include different relative proportions of polynucleotides from each of the two populations. For example, one population may make up about 0%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 100% of the total nucleotide content of a sample, and the other population making up the balance of the sample. Copies of and complements to the populations may then be made in accordance with the herein disclosure such as, for example, in a clustering process.

Oligonucleotides may then be hybridized to identifier sequences of copies of the populations of polynucleotides. Amount of hybridized oligonucleotide may be measured, such as, in an example where the oligonucleotides include a fluorophore, fluorescence emission may be detected and quantified, as a measure of total amount of oligonucleotide hybridized to identifier sequence of a given population. In such manner, amounts of oligonucleotide hybridized to each population may be measured and compared, to give an indication of relative abundance of copies of polynucleotides of each population following copying. In an example, a difference may be detectable when the sample included different relative proportions of each population of polynucleotide. For example, a difference may be detectable when one population made up about 0%, about 5%, about 10%, about 15%, about 20%, about 25%, about 30%, about 35%, about 40%, or about 45% of the nucleotide content of a sample before copying such as by clustering, and the other population made up the balance.

In an example, fluorescence emission is measured from oligonucleotides that include a fluorophore hybridized to identifier sequences of each population and a difference in fluorescence is ascertained. For example, oligonucleotides hybridizable to an identifier sequence of one populations of polynucleotides may include a detectably different fluorophore from an oligonucleotide hybridizable to an identifier sequence of another population of oligonucleotides such that fluorescence emission from one could be detected independently of fluorescence emission from the other and vice versa (e.g., Alexa 647, Alexa 532, etc.). In an example, fluorescence emitted from oligonucleotides hybridized to one identifier sequence may be at least about 10% more or less, or at least about 15% more or less, or at least about 20% more or less, or at least about 25% more or less, or at least about 30% more or less, or at least about 35% more or less, or at least about 40% more or less, or at least about 45% more or less, or at least about 50% more or less, than fluorescence emitted from oligonucleotides hybridized to another identifier sequence. In another example, fluorescence emitted from oligonucleotides hybridized to one identifier sequence may be about 10% more or less, or about 15% more or less, or about 20% more or less, or about 25% more or less, or about 30% more or less, or about 35% more or less, or about 40% more or less, or about 45% more or less, or about 50% more or less, than fluorescence emitted from oligonucleotides hybridized to another identifier sequence.

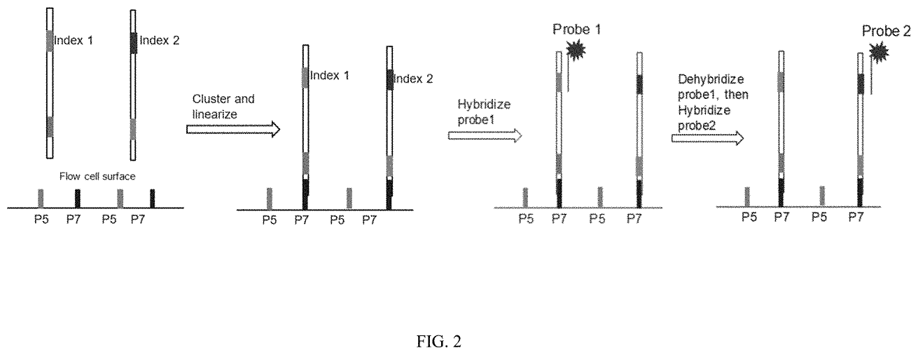

An example is shown in FIG. 2. In the far left panel, shown are two polynucleotides, one from each of two populations of polynucleotides. Each includes an index, or identifier sequence. In an actual example, a plurality of polynucleotides from each of the two or more populations may be used. A surface is shown on which solid-phase copying is to occur. In this example, the surface is that of a flow cell. Attached to the surface are primers (e.g., primers P5 and P7) to which a portion of the 3-prime ends of the polynucleotides are complementary and hybridizable. Polynucleotides are then hybridized to surface-attached primers, which primers are then extended by a polymerase to form a complement to the polynucleotides. Polynucleotides are then dehybridized, leaving surface-attached complements, extending from what had been surface-attached primers. The result is formation of surface-attached copies of polynucleotides of the populations, polymerized using surface-attached complements to the polynucleotides of the two or more populations as a template. Such strands are then linearized and dehybridized from each other, whereupon the process is repeated. By iteratively repeating this process, the number of surface-attached copies of polynucleotides of the two or more populations and surface-attached complements thereto is amplified, creating a surface-attached cluster. One set of strands, representing either copies of or complements to the polynucleotides of the population, may then be removed from the substrate such as by enzymatic cleavage. Refer to the arrow indicating “cluster and linearize” in FIG. 2.

If a feature or combination thereof that distinguishes one of the two or more populations from another causes bias in a copying process, or if differences in copying process influence, such bias may be reflected in a difference between amounts of surface attached copies of polynucleotides from one population as compared to another. Such differences may be ascertained by hybridizing an oligonucleotide probe, hybridizable to an identifier sequence of one population but not to an identifier sequence of any others and carrying a detectable attachment such as a fluorescent marker. As shown in the third panel of FIG. 2, such probe may be hybridized to copies of one population, excess, unbound probe washed away, then an amount of hybridized probe detected by measuring how much fluorescence is emitted following stimulation of the surface with a wavelength of electromagnetic radiation known to induce emission from the fluorescent marker attached to the oligonucleotide probe.

Subsequently, the first probe can be dehybridized and washed away, followed by hybridization with another probe. This other oligonucleotide is hybridizable to an identifier sequence of another population (but not to an identifier sequence of any others) and carries a detectable attachment such as a fluorescent marker. As shown in the last panel of FIG. 2, such other probe may be hybridized to copies of such other population, excess, unbound probe washed away, then an amount of hybridized probe detected by measuring how much fluorescence is emitted following stimulation of the surface with a wavelength of electromagnetic radiation known to induce emission from the fluorescent marker attached to said other oligonucleotide probe. Comparing how much fluorescence was detected from the first hybridized probe and from the second hybridized probe indicates a difference in how many copies of polynucleotides from the two of the two or more populations are attached to the surface.

By comparing this difference with a difference in amount of polynucleotides from each of the two or more populations of polynucleotides that were used to initiate the clustering process, an effect of one or more features distinguishing the two or more populations of polynucleotides on copying bias, or a bias resulting from a copying method, can be identified. That is, by comparing to each other an amount of each such oligonucleotide hybridized to copies of each of the two or more populations, normalized by a relative amount of polynucleotides from each population used for copying, the presence and magnitude of a given bias on copying may be ascertained. For example, if a feature causes a bias in copying, a relative amount of copies of polynucleotides from one of the two or more populations of polynucleotides characterized by said feature (such as a higher GC content, longer length, sample preparation process, any combination of two or more of the foregoing, etc.) may exceed that of copies of polynucleotides from another of the two or more populations of polynucleotides differentially characterized by said feature (such as a lower GC content, shorter length, different sample preparation process, or other different combination of two or more of the foregoing, etc.). In turn, detection of such a difference may indicate bias for or against copying polynucleotides characterized by said feature or combination of features.

The solid-state amplification process results in formation of copies of, and complements to, the polynucleotides from the initial populations bound to a surface. The copies of the polynucleotides include identifier sequences. In turn, the complements of said copies include complements to the identifier sequences, and said complements to the identifier sequences may also be uniquely hybridizable to an oligonucleotide probe that does not hybridize to other copies of or complements to polynucleotides attached to the surface. Hybridizing oligonucleotides to identifier sequences on surface-bound copies of polynucleotides and measuring the quantity of such hybridized oligonucleotides indicates how much copying of the polynucleotides occurred during the copying process. Similarly, hybridizing oligonucleotides to complements of identifier sequences on surface-bound complements of copies of polynucleotides and measuring the quantity of such hybridized oligonucleotides also indicates how much copying of the polynucleotides occurred during the copying process. Detection of amounts of either probe, hybridizable and hybridized to an identifier sequence of a surface-attached copy of a polynucleotide, or to a complement of an identifier sequence on a surface-attached complement, may be used as an indication of how much copying of a population of polynucleotides occurred.

Although in some examples polynucleotides from no two populations may have the same identifier sequence as each other, in other examples polynucleotides from two or more populations may include the same identifier sequence as each other. For example, polynucleotides may include more than one identifier sequence. polynucleotides from all of the two or more populations of polynucleotides may have a first identifier sequence that is distinct for each population. They may have a second identifier sequence that is shared between two or more of the two or more populations but different from any other of the two or more populations. Populations may have a third identifier sequence that is also shared by some populations but differentiates others. Populations may further have a fourth identifier sequence that is shared by all populations. In this example, differences between populations at a given identifier sequence may be such that an oligo that is hybridizable to one sequence at such hybridization sequence is not hybridizable to another sequence at such identifier sequence. The populations to which polynucleotides that surface-bound copies of and complements to are from may therefore be determined by hybridizing a probe specific for a given identifier sequence.

In a non-limiting example, there may be four populations of polynucleotides. Two populations may have a higher GC content than the other two populations, and two populations may have polynucleotides of a longer length than the other two populations. Length and GC content may be mixed between the four populations, with a first population having long polynucleotides with a high GC content, a second population having long polynucleotides with a low GC content, a third population having short polynucleotides with a high GC content, and a fourth population having short polynucleotides with a low GC content. Each population may have one, two, three, four, or more, identifier sequences. A first identifier sequence may be unique for each population. A second identifier sequence may differentiate between populations of differing lengths, with the first and second populations having the same second identifier sequence as each other and the third and fourth populations having the same identifier sequence as each other, with the second identifier sequence of the first and second populations being different than the second identifier sequence of the third and fourth populations. A third identifier sequence may differentiate between populations of different GC content, with the first and third populations having the same second identifier sequence as each other and the second and fourth populations having the same identifier sequence as each other, with the third identifier sequence of the first and third populations being different than the third identifier sequence of the second and fourth populations. A fourth identifier sequence may be shared by all populations.

After copying, hybridization of a probe specific for a sequence of a given identifier sequence and measurement of its hybridization may indicate different amounts of surface bound copies, i.e. how much copying occurred as to different populations or difference combinations of polynucleotides, according to the features that differentiate them or are shared by them. For example, an amount of each population individually may be determined by measuring hybridization of a probe specific for each sequence of the first hybridization sequence. An amount of copying of long and short polynucleotides may be determined by measuring hybridization of oligonucleotides to each sequence of the second identifier sequence. An amount of copying of high GC and low GC content polynucleotides may be determined by measuring hybridization of nucleotides to each sequence of the third identifier sequence. And a total amount of copying overall may be determined by measuring hybridization of nucleotides to the fourth identifier sequence. In other examples, more or fewer numbers of identifier sequences may be included, in some or all populations of polynucleotides, and combined in different manners across different populations. In other examples, there may be more than two sequences that may be present at a given identifier sequence, such as where several examples of a given feature are compared (e.g., low, medium, or high GC content, or short, medium and long polynucleotide length, etc.).

After clustering but before hybridization of oligonucleotides to identifier sequences, as described above copies of and complements to polynucleotides of the two or more populations are bound to a surface. It may be advantageous to remove the surface-bound complements before assessing hybridization to oligonucleotides to the identifier sequences. Or, in another example, it may be advantageous to remove surface-bound copies before measuring hybridization of oligonucleotides to complements of identifier sequences on surface-bound complements. Removal of surface-bound copies of or complements to polynucleotides of the two or more populations may be accomplished by including in the surface-bound primers from which such copies and complements extent a residue that can be selectively cleaved, removing the copies or complements that extend therefrom following clustering. For example, a primer may include a deoxyuridine (dU) moiety. Subsequent treatment with an enzyme formulation such as LMX1 can cleave the primer at the dU residue and release the polynucleotide extending therefrom. In another example, a surface-attached primer may include an 8-oxoguanine (oxo-G) residue. Subsequent treatment with an enzyme formulation such as LMX2 can cleave the primer at the oxo-G residue and release the polynucleotide extending therefrom.

Furthermore, aspects of a copying process such as a clustering process may be modified or compared to determine whether such aspect reduces, eliminates partially eliminates, worsens, or otherwise influences a bias that results from a feature of a population of polynucleotides. For example, if a feature that distinguishes polynucleotides from two or more different populations leads to, causes, or is determined to be associated with a feature according to a method as disclosed herein, copying, such as a clustering process, can be performed under different conditions and an effect such differences in copying conditions have on such bias may be determined. An aspect of a process by which copies of a population of polynucleotides is made (for example aspects of a clustering process) may be a feature, and such feature may differ between different populations. In an example, polynucleotides from two different populations, differing in a first feature (such as GC content, length, and/or a preparation process, as non-limiting examples), may be copied under each of two different conditions (a second feature). Any difference in an amount of copies of polynucleotides from the two populations when copied under one set of conditions, signifying that the first feature is related to a bias in copying, may then be compared to any difference in an amount of copies of polynucleotides from the two populations when copied under the other set of conditions. If such differences differ from each other, it would indicate that the difference in copying conditions owing to a bias related to the feature may be influenced by the conditions (that is, may be influenced by the second feature). In another example, a differentiating feature may be an aspect of a method of making copies of two or more populations of polynucleotides such as an aspect of a clustering process without other features also differing.

In an example, a feature-related bias reflected in a difference between how much two populations are copied when copied under one set of conditions may be less, or more (signified by a smaller or larger difference in an amount of copies between the two populations) than a feature-related bias reflected in a difference between how much two populations are copied when copied under another set of conditions. Any component, circumstance, environment, or other aspect under which copying occurs may be modified or tested for an effect on bias as reflected in differences in how much two populations of polynucleotides differentiated as to a feature are copied. For example, different polymerases, additives in a polymerization reaction (such as polyethylene glycol, salt, nucleotides, etc.), substrate, polymer coating of substrate, flow cell characteristics, temperature, timing, or number of polymerization cycles, components used for rehybridizing or linearizing copies of polynucleotides and complements thereto (e.g., LMX1 or LMX2, used in some biochemical processes after clustering to release a subset of surface-attached polynucleotides but before re-synthesizing surface-attached polynucleotides for a subsequent sequencing round), or any other condition, may be modified and compared. More than one example of a condition may be compared to more than one other. Also, multiple conditions may be modified to determine whether, for example, there is an interaction between them on a feature-related copying bias. Furthermore, a multiplicity of features may be compared for their individual and combined effects on bias as described above, and one or more conditions, alone, in combination, or both, may be tested for effects on any feature-related bias or biases.

A method according to the present disclosure provides advantages over other methods for assessing potential bias in clustering or other copying processes employed in a next generation sequencing technique. For example, as disclosed herein, sources of bias may be assessed without the need for sequencing copied polynucleotides. Potential sources of bias, and possible adjustments to minimize, eliminate, or otherwise affect bias, may be identified without having to proceed through the additional time, expense, and computational burden of performing and analyzing sequencing of polynucleotides. Furthermore, examples disclosed herein provide for high-throughput methods for assessing multiple possible sources of bias such as in the form of polynucleotide features, alone or in combination, as well as multiple variables whose modification may be brought to bear to eliminate or otherwise modify bias in copying such as conditions under which or according to which copying, e.g. clustering, occurs or conditions which otherwise may affect any aspect of polynucleotide copying that occurs prior to sequencing in an SBS process.

As to assessing bias attributable to GC content as a feature, populations of polynucleotides may be characterized by an average relative GC content. For example, certain species of microbes are known to have a relatively higher or lower average percentage of GC content than, for example, humans, whose genomes on average have an approximately equal proportion of GC and AT content. Some microbes, such as bacteria of the Rhodobacter group, are known to have elevated GC content, such as above 60% GC content. Others, such as Bacillus cereus, are known to have lower GC content, such as below 40% GC content. In an example, GC content may be a feature. A population of polynucleotides may be polynucleotides prepared from Rhodobacter, representing a higher GC content relative to other populations as a feature, another from humans, representing a medium GC content relative to other populations as a feature, or Bacillus cereus, representing a lower GC content relative to other populations as a feature. “higher” and “lower” here are used relatively. Thus, using human as a population, it could have higher GC or lower GC relative to another population as a feature, depending on GC content of such other population (e.g., Bacillus cereus and Rhodobacter, respectively).

In other examples, polynucleotides may be from synthetic or artificial sources with a predetermined GC content established by, for example, directly determining sequence of polynucleotides of the sample or stoichiometrically controlling incorporation of relative amounts of a given type of nucleotide in a strand, depending on its method of synthesis (e.g., using a template-independent method for sequence synthesis). A population of polynucleotides may include any intended or known percentage of GC, meaning total combined number of guanine and cytosine nucleobases out of total number of nucleobases (G, C, A, and T in total). A population may be defined by GC content as a characteristic of the population as a whole, even if individual polynucleotides of the population may have a GC content that differs from the GC content of the population as a whole.

A population may have about 5% GC content, about 10% GC content, about 15% GC content, about 20% GC content, about 25% GC content, about 30% GC content, about 35% GC content, about 40% GC content, about 45% GC content, about 50% GC content, about 55% GC content, about 60% GC content, about 65% GC content, about 70% GC content, about 75% GC content, about 80% GC content, about 85% GC content, or about 90% GC content, or any intervening amount of GC content. All other possible comparisons are explicitly included as aspects of the present disclosure.

As to assessing bias attributable to polynucleotide length, populations of polynucleotides may be characterized by an average relative polynucleotide length. For example, nucleic acid molecules may be isolated from a sample, such as a cell or other biological source, and fragmented during sample preparation by any of various methods. By adjusting the parameters used in fragmentation methods such as sonication time, polynucleotides of various lengths can be created. Polynucleotides of a desired length may then be isolated from the resulting fragments. A population may be defined by polynucleotide length as a characteristic of the population as a whole, even if individual polynucleotides of the population may have a length that differs from the polynucleotide length of the population as so determined. In another example, polynucleotide length as a feature of a population may be predetermined by polymerizing polynucleotides of a predetermined length from a designed template.

A population of polynucleotides may have a length of about 100 nucleotides, about 150 nucleotides, about 200 nucleotides, about 250 nucleotides, about 300 nucleotides, about 350 nucleotides, about 400 nucleotides, about 450 nucleotides, about 500 nucleotides, about 550 nucleotides, about 600 nucleotides, about 650 nucleotides, about 700 nucleotides, about 750 nucleotides, about 800 nucleotides, about 850 nucleotides, about 900 nucleotides, about 950 nucleotides, about 1,000 nucleotides, about 1,050 nucleotides, about 1,200 nucleotides, about 1,250 nucleotides, about 1,300 nucleotides, about 1,350 nucleotides, about 1,400 nucleotides, about 1,450 nucleotides, about 1,500 nucleotides, about 1,550 nucleotides, about 1,600 nucleotides, about 1,650 nucleotides, about 1,700 nucleotides, about 1,750 nucleotides, about 1,800 nucleotides, about 1,850 nucleotides, about 1,900 nucleotides, about 1,950 nucleotides, about 2000 nucleotides, or longer.

A feature may also include other aspects of population preparation, such as nucleotide populations (DNA libraries) prepared by different library prep methods or library prep kits from different vendors. Effects of aspects of a clustering process may also be compared to determine whether and how such conditions affect a bias or hypothesized bias related to feature. Each of two or more population of polynucleotides, differentiated from each other by one or more features, may be subjected to one, two, or more copying processes, such as clustering processes, with a condition varying between the two or more processes. Whether a copying bias results from the feature difference under one condition and/or the other, or whether the amount or presence of the bias differs depending on which copying condition it was subjected to, may indicate whether a bias related to the feature may be altered by so modifying the condition. In an example, multiple conditions may be modified, or several different examples of a condition may be compared. Examples of conditions that may be modified include components of a reaction solution used for solid-state copying, such as a clustering process (such as polymerase used, pH, concentration of polynucleotide or any component, linearizing enzyme used such as LMX1 or LMX2 or other, performance additives included such as GP32 or UvsX or other nucleotide binding protein, polyethylene glycol, creatine phosphate, or other additive, type of substrate surface, or type, presence, or thickness of polymer coating of surface on which solid-phase PCR, or clustering, occurs), number or duration of rounds of copying, temperature, etc. In some examples, any aspect of a method of preparation of a population of polynucleotides, and/or copying method such as during cluster formation from a population of polynucleotides, may be modified and evaluated according to the method disclosed herein to determine possibility of bias.

Non-limiting examples of parameters that may be varied, as a feature, in a reagent used in a method of making copies (e.g., ExAmp, bridge, or other clustering process) include various enzyme concentrations (and ratios), additives (concentrations and ratios), solution pH, polynucleotide concentration included in a solution for copying, nucleotide concentration included in a solution for copying.

Non-limiting examples of temperature at which copying occurs (e.g., at, above, or below, in an example, about 20 degrees Celsius), duration of polymerization or wash steps, to reagent replenishment method or duration, flow rate of reagent into flowcell or other substrate used, etc., may be modified and interrogated accordingly. Clustering time may be varied as a feature (e.g., for less than about 30 min, or within about 30 min to about an hour, or within from about one to about two hours, or within from about two to about three hours, or within from about three to about four hours, or within from about four hours to about five hours, or within from about five hours to about six hours, or within from about six hours to about seven hours, or within from about seven hours to about eight hours, or within from about eight hours to about nine hours, or within from about nine hours to about ten hours, or within from about ten hours to about eleven hours, or within from about eleven hours to about twelve hours, or within about twelve hours to about twenty four hours, or within about twenty four hours to about thirty six hours, or within about thirty six hours to about forty eight hours, or within about forty eight hours to about seventy two hours, or longer). Durations of each aspect of a copying or clustering process such as duration of incubation of reagents in solution may be varied as a feature (e.g., for about 10 sec, or about 20 sec, or about 30 sec, or about 40 sec, or about 50 sec, or about 60 sec, or about 70 sec, or about 80 sec, or about 90 sec, or about 100 sec, or about 110 sec, or about 120 sec, or longer).

Fluidic speed, or the rate at which reagent flows through a flow cell, may be varied as a feature (e.g., flow rate may be about 10 ul/min, or about 20 ul/min, or about 30 ul/min, or about 40 ul/min, or about 50 ul/min, or about 60 ul/min, or about 70 ul/min, or about 80 ul/min, or about 90 ul/min, or about 100 ul/min, or about 110 ul/min, or about 120 ul/min, or about 130 ul/min, or about 140 ul/min, or about 150 ul/min, or at a higher rate). Other aspects that could be modified as features include pH (e.g., above or below, or at, about pH 7.5), type of buffer (e.g., Tris-based or other), and concentration of buffer or other component or components of a clustering solution (e.g., about 100 nM, or less or more than about 100 nM).

In an example, polynucleotides from two or more populations may be combined in a solution and the solution added to a substrate such as a flow cell for copying, including, for example, copying by a clustering process. Different identifier sequences on polynucleotides of the two or more populations permit identifying the population surface-attached copies are copies of. In an example, a relative proportion of the total amount of polynucleotides added accounted for by polynucleotides from a population may be controlled, and varied across several different solutions. For example, a total number of polynucleotides in a solution added to a flow cell, or a lane of a flow cell, may have about equal proportions of polynucleotides from each of two populations of polynucleotides. A total number of polynucleotides in another solution added to a flow cell, or a lane of a flow cell, may have about 25% of polynucleotides from one population of polynucleotides and about 75% from another, whereas a total number of polynucleotides in yet another solution added to a flow cell, or a lane of a flow cell, may have about 75% of polynucleotides from the other population of polynucleotides and about 25% from the one. Any other split between proportions of polynucleotides from one and the other population may be used in different solutions (e.g., about 5%/95%, about 10%/90%, about 15%/85%, about 20%/80%, about 25%/75%, about 30%/70%, about 35%/65%, about 40%/60%, about 45%/55%, about 50%/50%, about 55%/45%, about 60%/40%, about 65%/35%, about 70%/30%, about 75%/25%, about 80%/20%, about 85%/15%, about 90%/10%, about 95%/5%, or any intervening relative proportions).

Library Preparation

Libraries including polynucleotides may be prepared in any suitable manner to attach oligonucleotide adapters to target polynucleotides. As used herein, a “library” is a population of polynucleotides from a given source or sample. A library includes a plurality of target polynucleotides. As used herein, a “target polynucleotide” is a polynucleotide that is desired for inclusion in a copying process such as a clustering process. The target polynucleotide may be essentially any polynucleotide of known or unknown sequence. It may be, for example, a fragment of genomic DNA or cDNA. The target polynucleotides may be derived from a primary polynucleotide sample that has been randomly fragmented. The target polynucleotides may be processed into templates suitable for amplification by the placement of primer sequences at the ends of each target fragment, such as identifier sequences, sequences complementary to surface attached primers, etc. The target polynucleotides may also be obtained from a primary RNA sample by reverse transcription into cDNA.

As used herein, the terms “polynucleotide” and “oligonucleotide” may be used interchangeably and refer to a molecule including two or more nucleotide monomers covalently bound to one another, typically through a phosphodiester bond. Polynucleotides typically contain more nucleotides than oligonucleotides. For purposes of illustration and not limitation, a polynucleotide may be considered to contain 15, 20, 30, 40, 50, 100, 200, 300, 400, 500, or more nucleotides, while an oligonucleotide may be considered to contain 100, 50, 20, 15, or fewer nucleotides.

Polynucleotides and oligonucleotides may include deoxyribonucleic acid (DNA) or ribonucleic acid (RNA). The terms should be understood to include, as equivalents, analogs of either DNA or RNA made from nucleotide analogs and to be applicable to single stranded (such as sense or antisense) and double stranded polynucleotides. The term as used herein also encompasses cDNA, that is complementary or copy DNA produced from an RNA template, for example by the action of reverse transcriptase.

Primary polynucleotide molecules may originate in double-stranded DNA (dsDNA) form (e.g. genomic DNA fragments, PCR and amplification products and the like) or may have originated in single-stranded form, as DNA or RNA, and been converted to dsDNA form. By way of example, mRNA molecules may be copied into double-stranded cDNAs using standard techniques well known in the art. The precise sequence of primary polynucleotides is generally not material to the disclosure presented herein, and may be known or unknown.

In some examples, the primary target polynucleotides are RNA molecules. In an aspect of such examples, RNA isolated from specific samples is first converted to double-stranded DNA using techniques known in the art. The double-stranded DNA may then be index tagged with a library specific tag. Different preparations of such double-stranded DNA including library specific index tags may be generated, in parallel, from RNA isolated from different sources or samples. Subsequently, different preparations of double-stranded DNA including different library specific index tags may be mixed, copied en masse, and the identity of each sequenced fragment determined with respect to the population from which it was isolated/derived by virtue of the presence of a library specific index tag sequence.

In some examples, the primary target polynucleotides are DNA molecules. For example, the primary polynucleotides may represent the entire genetic complement of an organism, and are genomic DNA molecules, such as human DNA molecules, which include both intron and exon sequences (coding sequence), as well as non-coding regulatory sequences such as promoter and enhancer sequences. Although it could be envisaged that particular sub-sets of polynucleotide sequences or genomic DNA could also be used, such as, for example, particular chromosomes or a portion thereof. In many examples, the sequence of the primary polynucleotides is not known. The DNA target polynucleotides may be treated chemically or enzymatically either prior to, or subsequent to a fragmentation processes, such as a random fragmentation process, and prior to, during, or subsequent to the ligation of the adapter oligonucleotides.

In one example, the primary target polynucleotides are fragmented to appropriate lengths suitable for sequencing. The target polynucleotides may be fragmented in any suitable manner. Preferably, the target polynucleotides are randomly fragmented. Random fragmentation refers to the fragmentation of a polynucleotide in a non-ordered fashion by, for example, enzymatic, chemical or mechanical means. Any suitable fragmentation methods may be employed. For the sake of clarity, generating smaller fragments of a larger piece of polynucleotide via specific PCR amplification of such smaller fragments is not equivalent to fragmenting the larger piece of polynucleotide because the larger piece of polynucleotide remains in intact, i.e., is not fragmented by the PCR amplification (though a method as disclosed herein may be performed on populations of polynucleotides created by either technique). Moreover, random fragmentation is designed to produce fragments irrespective of the sequence identity or position of nucleotides including and/or surrounding the break.

In some examples, the random fragmentation is by mechanical means such as nebulization or sonication to produce fragments of about 50 base pairs in length to about 1500 base pairs in length, such as 50-700 base pairs in length or 50-500 base pairs in length.

Fragmentation of polynucleotide molecules by mechanical means (nebulization, sonication and Hydroshear for example) may result in fragments with a heterogeneous mix of blunt and 3-prime- and 5-prime-overhanging ends. Fragment ends may be repaired using methods or kits (such as the Lucigen DNA terminator End Repair Kit) known in the art to generate ends that are optimal for insertion, for example, into blunt sites of cloning vectors. In some examples, the fragment ends of the population of nucleic acids are blunt ended. The fragment ends may be blunt ended and phosphorylated. The phosphate moiety may be introduced via enzymatic treatment, for example, using polynucleotide kinase.

In some examples, the target polynucleotide sequences are prepared with single overhanging nucleotides by, for example, activity of certain types of DNA polymerase such as Taq polymerase or Klenow exo minus polymerase which has a nontemplate-dependent terminal transferase activity that adds a single deoxynucleotide, for example, deoxyadenosine (A) to the 3-prime ends of, for example, PCR products. Such enzymes may be utilized to add a single nucleotide ‘A’ to the blunt ended 3-prime terminus of each strand of the target polynucleotide duplexes. Thus, an ‘A’ could be added to the 3-prime terminus of each end repaired duplex strand of the target polynucleotide duplex by reaction with Taq or Klenow exo minus polymerase, while the adapter polynucleotide construct could be a T-construct with a compatible ‘T’ overhang present on the 3-prime terminus of each duplex region of the adapter construct. This end modification also prevents self-ligation of the target polynucleotides such that there is a bias towards formation of the combined ligated adapter-target polynucleotides.

In some examples, fragmentation is accomplished through tagmentation. In such methods transposases are employed to fragment a double stranded polynucleotide and attach a universal primer sequence into one strand of the double stranded polynucleotide. The resulting molecule may be gap-filled and subject to extension, for example by PCR amplification, using primers that include a 3-prime end having a sequence complementary to the attached universal primer sequence and a 5-prime end that contains other sequences of an adapter.

The adapters may be attached to the target polynucleotide in any other suitable manner. In some examples, the adapters may be introduced in a single-step process. In some examples, the adapters may be introduced in a multi-step process, such as a two-step process, involving ligation of a portion of the adapter to the target polynucleotide having a universal primer sequence. The second step includes extension, for example by PCR amplification, using primers that include a 3-prime end having a sequence complementary to the attached universal primer sequence and a 5-prime end that contains other sequences of an adapter. Additional extensions may be performed to provide additional sequences to the 5-prime end of the resulting previously extended polynucleotide.

In some examples, the entire adapter is ligated to the fragmented target polynucleotide. Preferably, the ligated adapter includes a double stranded region that is ligated to a double stranded target polynucleotide. Preferably, the double-stranded region is as short as possible without loss of function. In this context, “function” refers to the ability of the double-stranded region to form a stable duplex under standard reaction conditions. In some examples, standard reactions conditions refer to reaction conditions for an enzyme-catalyzed polynucleotide ligation reaction (e.g. incubation at a temperature in the range of 4° C. to 25° C. in a ligation buffer appropriate for the enzyme), such that the two strands forming the adapter remain partially annealed during ligation of the adapter to a target molecule. Ligation methods utilize ligase enzymes such as DNA ligase to effect or catalyze joining of the ends of the two polynucleotide strands of, in this case, the adapter duplex oligonucleotide and the target polynucleotide duplexes, such that covalent linkages are formed. The adapter duplex oligonucleotide may contain a 5-prime-phosphate moiety in order to facilitate ligation to a target polynucleotide 3-prime-OH. The target polynucleotide may contain a 5-prime-phosphate moiety, either residual from the shearing process, or added using an enzymatic treatment step, and has been end repaired, and optionally extended by an overhanging base or bases, to give a 3-prime-OH suitable for ligation. In this context, attaching means covalent linkage of polynucleotide strands which were not previously covalently linked. In an aspect, such attaching takes place by formation of a phosphodiester linkage between the two polynucleotide strands, but other means of covalent linkage (e.g. non-phosphodiester backbone linkages) may be used.

Any suitable adapter may be attached to a target polynucleotide via any suitable process, such as those discussed above. The adapter includes a library-specific index tag sequence. The index tag sequence may be attached to the target polynucleotides from each library before the sample is immobilized for sequencing. The index tag is not itself formed by part of the target polynucleotide, but becomes part of the template for amplification. The index tag may be a synthetic sequence of nucleotides which is added to the target as part of the template preparation step. Accordingly, a library-specific index tag is a nucleic acid sequence tag which is attached to each of the target molecules of a particular library, the presence of which is indicative of or is used to identify the library from which the target molecules were isolated.

Preferably, the index tag sequence is 20 nucleotides or less in length. For example, the index tag sequence may be 1-10 nucleotides or 4-6 nucleotides in length. A four nucleotide index tag gives a possibility of multiplexing 256 samples on the same array, a six base index tag enables 4,096 samples to be processed on the same array.

Adapters may contain more than one index tag (or identifier sequence) so that the multiplexing possibilities may be increased.

Adapters may include a double stranded region and a region including two non-complementary single strands. The double-stranded region of the adapter may be of any suitable number of base pairs. Preferably, the double stranded region is a short double-stranded region, typically including 5 or more consecutive base pairs, formed by annealing of two partially complementary polynucleotide strands. This “double-stranded region” of the adapter refers to a region in which the two strands are annealed and does not imply any particular structural conformation. In some examples, the double stranded region includes 20 or less consecutive base pairs, such as 10 or less or 5 or less consecutive base pairs.

The stability of a double-stranded region may be increased, and hence its length potentially reduced, by inclusion of non-natural nucleotides which exhibit stronger base-pairing than standard Watson-Crick base pairs. Two strands of an adapter may be 100% complementary in a double-stranded region.

When an adapter is attached to the target polynucleotide, the non-complementary single stranded region may form the 5-prime and 3-prime ends of the polynucleotide to be sequenced. The term “non-complementary single stranded region” refers to a region of the adapter where the sequences of the two polynucleotide strands forming the adapter exhibit a degree of non-complementarity such that the two strands are not capable of fully annealing to each other under standard annealing conditions for a PCR reaction.

The non-complementary single stranded region is provided by different portions of the same two polynucleotide strands which form the double-stranded region. The lower limit on the length of the single-stranded portion will typically be determined by function of, for example, providing a suitable sequence for binding of a primer for primer extension, PCR and/or sequencing. Theoretically there is no upper limit on the length of the unmatched region, except that in general it is advantageous to minimize the overall length of the adapter, for example, in order to facilitate separation of unbound adapters from adapter-target constructs following the attachment step or steps. Therefore, it is generally preferred that the non-complementary single-stranded region of the adapter is 50 or less consecutive nucleotides in length, such as 40 or less, 30 or less, or 25 or less consecutive nucleotides in length.

The library-specific index tag sequence may be located in a single-stranded, double-stranded region, or span the single-stranded and double-stranded regions of the adapter. Preferably, the index tag sequence is in a single-stranded region of the adapter.

The adapters may include any other suitable sequence in addition to the index tag sequence. For example, the adapters may include universal extension primer sequences, which are typically located at the 5-prime or 3-prime end of the adapter and the resulting polynucleotide for sequencing. The universal extension primer sequences may hybridize to complementary primers bound to a surface of a solid substrate. The complementary primers include a free 3-prime end from which a polymerase or other suitable enzyme may add nucleotides to extend the sequence using the hybridized library polynucleotide as a template, resulting in a reverse strand of the library polynucleotide being coupled to the solid surface. Such extension may be part of a sequencing run or cluster amplification.

In some examples, the adapters include one or more universal sequencing primer sequences. The universal sequencing primer sequences may bind to sequencing primers to allow sequencing of an index tag sequence, a target sequence, or an index tag sequence and a target sequence.

The precise nucleotide sequence of the adapters is generally not material and may be selected by the user such that the desired sequence elements are ultimately included in the common sequences of the library of templates derived from the adapters to, for example, provide binding sites for particular sets of universal extension primers and/or sequencing primers.

The adapter oligonucleotides may contain exonuclease resistant modifications such as phosphorothioate linkages.

Preferably, the adapter is attached to both ends of a target polypeptide to produce a polynucleotide having a first adapter-target-second adapter sequence of nucleotides. The first and second adapters may be the same or different. If the first and second adapters are different, at least one of the first and second adapters includes a library-specific identifier sequence.

“First adapter-target-second adapter sequence” or an “adapter-target-adapter” sequence refers to the orientation of the adapters relative to one another and to the target and does not necessarily mean that the sequence may not include additional sequences, such as linker sequences, for example.

Other libraries may be prepared in a similar manner, each including at least one library-specific index tag sequence or combinations of index tag sequences different than an index tag sequence or combination of index tag sequences from the other libraries.

As used herein, “attached” or “bound” are used interchangeably in the context of an adapter relative to a target sequence. As described above, any suitable process may be used to attach an adapter to a target polynucleotide. For example, the adapter may be attached to the target through ligation with a ligase; through a combination of ligation of a portion of an adapter and addition of further or remaining portions of the adapter through extension, such as PCR, with primers containing the further or remaining portions of the adapters; through transposition to incorporate a portion of an adapter and addition of further or remaining portions of the adapter through extension, such as PCR, with primers containing the further or remaining portions of the adapters; or the like. Preferably, the attached adapter oligonucleotide is covalently bound to the target polynucleotide.

After the adapters are attached to the target polynucleotides, the resulting polynucleotides may be subjected to a clean-up process to enhance the purity to the adapter-target-adapter polynucleotides by removing at least a portion of the unincorporated adapters. Any suitable clean-up process may be used, such as electrophoresis, size exclusion chromatography, or the like. In some examples, solid phase reverse immobilization (SPRI) paramagnetic beads may be employed to separate the adapter-target-adapter polynucleotides from the unattached adapters. While such processes may enhance the purity of the resulting adapter-target-adapter polynucleotides, some unattached adapter oligonucleotides likely remain.

Methods for amplifying immobilized adapter-target-adapter molecules include, but are not limited to, bridge amplification and kinetic exclusion. Amplification can be carried out using one or more immobilized primers. The immobilized primer(s) can be a lawn on a planar surface.

The term “solid-phase amplification” as used herein refers to any nucleic acid amplification reaction carried out on or in association with a solid support such that all or a portion of the amplified products are immobilized on the solid support as they are formed. In particular, the term encompasses solid-phase polymerase chain reaction (solid-phase PCR) and solid phase isothermal amplification which are reactions analogous to standard solution phase amplification, except that one or both of the forward and reverse amplification primers is/are immobilized on the solid support. Solid phase PCR covers systems such as emulsions, wherein one primer is anchored to a bead and the other is in free solution, and colony formation in solid phase gel matrices wherein one primer is anchored to the surface, and one is in free solution.

In some examples, the solid support includes a patterned surface. A “patterned surface” refers to an arrangement of different regions in or on an exposed layer of a solid support. The term flow cell “support” or “substrate” refers to a support or substrate upon which surface chemistry may be added. The term “patterned substrate” refers to a support in which or on which depressions are defined. The term “non-patterned substrate” refers to a substantially planar support. The substrate may also be referred to herein as a “support,” “patterned support,” or “non-patterned support.” The support may be a wafer, a panel, a rectangular sheet, a die, or any other suitable configuration. The support is generally rigid and is insoluble in an aqueous liquid. The support may be inert to a chemistry that is used to modify the depressions. For example, a support can be inert to chemistry used to form a polymer coating layer, to attach primers such as to a polymer coating layer that has been deposited, etc. Examples of suitable supports include epoxy siloxane, glass and modified or functionalized glass, polyhedral oligomeric silsequioxanes (POSS) and derivatives thereof, plastics (including acrylics, polystyrene and copolymers of styrene and other materials, polypropylene, polyethylene, polybutylene, polyurethanes, polytetrafluoroethylene (such as TEFLON® from Chemours), cyclic olefins/cyclo-olefin polymers (COP) (such as ZEONOR® from Zeon), polyimides, etc.), nylon, ceramics/ceramic oxides, silica, fused silica, or silica-based materials, aluminum silicate, silicon and modified silicon (e.g., boron doped p+ silicon), silicon nitride (Si₃N₄), silicon oxide (SiO₂), tantalum pentoxide (TaO₅) or other tantalum oxide(s) (TaOₓ), hafnium oxide (HaO₂), carbon, metals, inorganic glasses, or the like. The support may also be glass or silicon or a silicon-based polymer such as a POSS material, optionally with a coating layer of tantalum oxide or another ceramic oxide at the surface. A POSS material may be that disclosed in Kejagoas et al., Microelectronic Engineering 86 (2009) 776-668, which is incorporated by reference herein in its entirety.

In an example, depressions may be wells such that the patterned substrate includes an array of wells in a surface thereof. The wells may be micro wells or nanowells. The size of each well may be characterized by its volume, well opening area, depth, and/or diameter. For example, one or more of the regions can be portions where one or more amplification primers are present. The portions can be separated by interstitial regions where amplification primers are not present. In some examples, the pattern can be an x-y format of features that are in rows and columns. In some examples, the pattern can be a repeating arrangement of portions and/or interstitial regions. In some examples, the pattern can be a random arrangement of portions and/or interstitial regions.

In some examples, the solid support includes an array of wells or depressions in a surface. This may be fabricated using a variety of techniques, including, but not limited to, photolithography, stamping techniques, molding techniques and microetching techniques. The technique used may depend on the composition and shape of the array substrate.

The features in a patterned surface can be wells in an array of wells (e.g. microwells or nanowells) on glass, silicon, plastic or other suitable solid supports with patterned, covalently-linked gel such as poly(N-(5-azidoacetamidylpentyl)acrylamide-co-acrylamide) (PAZAM). The process creates gel pads used for sequencing that can be stable over sequencing runs with a large number of cycles. The covalent linking of the polymer to the wells is helpful for maintaining the gel in the structured features throughout the lifetime of the structured substrate during a variety of uses. However in many examples, the gel need not be covalently linked to the wells. For example, in some conditions silane free acrylamide that is not covalently attached to any part of the structured substrate, can be used as the gel material.

In some examples, a structured substrate can be made by patterning a solid support material with wells (e.g. microwells or nanowells), coating the patterned support with a gel material (e.g. PAZAM, SFA or chemically modified variants thereof, such as the azidolyzed version of SFA (azido-SFA)) and polishing the gel coated support, for example via chemical or mechanical polishing, thereby retaining gel in the wells but removing or inactivating substantially all of the gel from the interstitial regions on the surface of the structured substrate between the wells. Primer nucleic acids can be attached to gel material. A solution of target nucleic acids (e.g. a fragmented human genome) can then be contacted with the polished substrate such that individual target nucleic acids may seed individual wells via interactions with primers attached to the gel material; however, the target nucleic acids will not occupy the interstitial regions due to absence or inactivity of the gel material. Amplification of the target nucleic acids will be confined to the wells since absence or inactivity of gel in the interstitial regions prevents outward migration of the growing nucleic acid colony. The process is conveniently manufacturable, being scalable and utilizing conventional micro- or nanofabrication methods.

The disclosed subject matter includes as an example “solid-phase” amplification methods in which only one amplification primer is immobilized (the other primer being present, for example, in free solution), in other examples the solid support may be provided with both the forward and the reverse primers immobilized. Some examples include a “plurality” of identical forward primers and/or a “plurality” of identical reverse primers immobilized on a solid support, since an amplification process may include an excess of primers to sustain amplification. References herein to forward and reverse primers are to be interpreted accordingly as encompassing a “plurality” of such primers unless the context indicates otherwise.

Any given amplification reaction includes at least one type of forward primer and at least one type of reverse primer specific for the template to be amplified. However, in certain examples forward and reverse primers may include template-specific portions of identical sequence, and may have entirely identical nucleotide sequence and structure (including any non-nucleotide modifications). In other words, it is possible to carry out solid-phase amplification using only one type of primer, and such single-primer methods are encompassed within the scope of this disclosure. Other examples may use forward and reverse primers which contain identical template-specific sequences but which differ in some other structural features. For example one type of primer may contain a non-nucleotide modification which is not present in the other.

The terms “cluster” and “colony” are used interchangeably herein to refer to a discrete site on a solid support including a plurality of identical immobilized nucleic acid strands and a plurality of identical immobilized complementary nucleic acid strands. The term “clustered array” refers to an array formed from such clusters or colonies. In this context the term “array” is not to be understood as requiring an ordered arrangement of clusters.

The term “solid phase”, or “surface”, is used to mean either a planar array wherein primers are attached to a flat surface, for example, glass, silica or plastic microscope slides or similar flow cell devices; beads, wherein either one or two primers are attached to the beads and the beads are amplified; or an array of beads on a surface after the beads have been amplified.

Clustered arrays can be prepared using either a process of thermocycling or a process whereby the temperature is maintained as a constant, and the cycles of extension and denaturing are performed using changes of reagents. In an example, an isothermal process may advantageously include use of a lower temperature.

It will be appreciated that any of the amplification methodologies described herein or generally known in the art may be utilized with universal or target-specific primers to amplify immobilized DNA fragments. Suitable methods for amplification include, but are not limited to, the polymerase chain reaction (PCR), strand displacement amplification (SDA), transcription mediated amplification (TMA) and nucleic acid sequence based amplification (NASBA). The above amplification methods may be employed to amplify one or more nucleic acids of interest. For example, PCR, including multiplex PCR, SDA, TMA, NASBA and the like may be utilized to amplify immobilized DNA fragments. In some examples, primers directed specifically to the polynucleotide of interest are included in the amplification reaction.

Other suitable methods for amplification of polynucleotides may include oligonucleotide extension and ligation, rolling circle amplification (RCA), or oligonucleotide ligation assay (OLA) technologies. It will be appreciated that these amplification methodologies may be designed to amplify immobilized DNA fragments. For example, in some examples, an amplification method may include ligation probe amplification or oligonucleotide ligation assay (OLA) reactions that contain primers directed specifically to the nucleic acid of interest. In some examples, the amplification method may include a primer extension-ligation reaction that contains primers directed specifically to the nucleic acid of interest. As a non-limiting example of primer extension and ligation primers that may be specifically designed to amplify a nucleic acid of interest, the amplification may include primers used for the GoldenGate assay (Illumina, Inc., San Diego, Calif.).

Exemplary isothermal amplification methods that may be used in a method of the present disclosure include, but are not limited to, Multiple Displacement Amplification (MDA) or isothermal strand displacement nucleic acid amplification. Other non-PCR-based methods that may be used in the present disclosure include, for example, strand displacement amplification (SDA) or hyper-branched strand displacement amplification. Isothermal amplification methods may be used with the strand-displacing Phi 29 polymerase or Bst DNA polymerase large fragment, 5-prime->3-prime exo—for random primer amplification of genomic DNA. The use of these polymerases takes advantage of their high processivity and strand displacing activity. High processivity allows the polymerases to produce fragments that are 10-20 kb in length. As set forth above, smaller fragments may be produced under isothermal conditions using polymerases having low processivity and strand-displacing activity such as Klenow polymerase.

DNA polymerases may include those that have been classified by structural homology into families identified as A, B, C, D, X, Y, and RT. DNA Polymerases in Family A include, for example, T7 DNA polymerase, eukaryotic mitochondrial DNA Polymerase gamma., E. coli DNA Pol I (including Klenow fragment), Thermus aquaticus Pol I, and Bacillus stearothermophilus Pol I. DNA Polymerases in Family B include, for example, eukaryotic DNA polymerases a, 6, and E; DNA polymerase C; T4 DNA polymerase, Phi29 DNA polymerase, Thermococcus sp. 9° N-7 archaeon polymerase (also known as 9° N™) and variants thereof such as examples disclosed in U.S. Patent Application Publication No. 2016/0032377 A1, and RB69 bacteriophage DNA polymerase. Family C includes, for example, the E. coli DNA Polymerase III alpha subunit. Family D includes, for example, polymerases derived from the Euryarchaeota subdomain of Archaea. DNA Polymerases in Family X include, for example, eukaryotic polymerases Pol beta, Pol sigma, Pol lambda, and Pol mu, and S. cerevisiae Po14. DNA Polymerases in Family Y include, for example, Pol eta, Pol iota, Pol kappa, E. coli Pol IV (DINB) and E. coli Pol V (UmuD′2C). The RT (reverse transcriptase) family of DNA polymerases includes, for example, retrovirus reverse transcriptases and eukaryotic telomerases. Example RNA polymerases include, but are not limited to, viral RNA polymerases such as T7 RNA polymerase; Eukaryotic RNA polymerases such as RNA polymerase I, RNA polymerase II, RNA polymerase III, RNA polymerase IV, and RNA polymerase V; and Archaea RNA polymerase. Other polymerases are also included among polymerases as referred to herein, as are any other functional polymerases including those having sequences modified by comparison to any of the above mentioned polymerase enzymes, which are provided merely as a listing of non-limiting examples.

In some examples, isothermal amplification can be performed using kinetic exclusion amplification (KEA), also referred to as exclusion amplification (ExAmp). A nucleic acid library of the present disclosure can be made using a method that includes a step of reacting an amplification reagent to produce a plurality of amplification sites that each includes a substantially clonal population of amplicons from an individual target nucleic acid that has seeded the site. In some examples the amplification reaction proceeds until a sufficient number of amplicons are generated to fill the capacity of the respective amplification site. Filling an already seeded site to capacity in this way inhibits target nucleic acids from landing and amplifying at the site thereby producing a clonal population of amplicons at the site. In some examples, apparent clonality can be achieved even if an amplification site is not filled to capacity prior to a second target nucleic acid arriving at the site. Under some conditions, amplification of a first target nucleic acid can proceed to a point that a sufficient number of copies are made to effectively outcompete or overwhelm production of copies from a second target nucleic acid that is transported to the site. For example in an example that uses a bridge amplification process on a circular feature that is smaller than 500 nm in diameter, it has been determined that after 14 cycles of exponential amplification for a first target nucleic acid, contamination from a second target nucleic acid at the same site will produce an insufficient number of contaminating amplicons to adversely impact sequencing-by-synthesis analysis on an Illumina sequencing platform.

In some examples, kinetic exclusion can occur when a process occurs at a sufficiently rapid rate to effectively exclude another event or process from occurring. Take for example the making of a nucleic acid array where sites of the array are randomly seeded with target nucleic acids from a solution and copies of the target nucleic acid are generated in an amplification process to fill each of the seeded sites to capacity. In accordance with the kinetic exclusion methods of the present disclosure, the seeding and amplification processes can proceed simultaneously under conditions where the amplification rate exceeds the seeding rate. As such, the relatively rapid rate at which copies are made at a site that has been seeded by a first target nucleic acid will effectively exclude a second nucleic acid from seeding the site for amplification.

Kinetic exclusion can exploit a relatively slow rate for initiating amplification (e.g. a slow rate of making a first copy of a target nucleic acid) vs. a relatively rapid rate for making subsequent copies of the target nucleic acid (or of the first copy of the target nucleic acid). In the example of the previous paragraph, kinetic exclusion occurs due to the relatively slow rate of target nucleic acid seeding (e.g. relatively slow diffusion or transport) vs. the relatively rapid rate at which amplification occurs to fill the site with copies of the nucleic acid seed. In another example, kinetic exclusion can occur due to a delay in the formation of a first copy of a target nucleic acid that has seeded a site (e.g. delayed or slow activation) vs. the relatively rapid rate at which subsequent copies are made to fill the site. In this example, an individual site may have been seeded with several different target nucleic acids (e.g. several target nucleic acids can be present at each site prior to amplification). However, first copy formation for any given target nucleic acid can be activated randomly such that the average rate of first copy formation is relatively slow compared to the rate at which subsequent copies are generated. In this case, although an individual site may have been seeded with several different target nucleic acids, kinetic exclusion will allow only one of those target nucleic acids to be amplified. More specifically, once a first target nucleic acid has been activated for amplification, the site will rapidly fill to capacity with its copies, thereby preventing copies of a second target nucleic acid from being made at the site.

An amplification reagent can include further components that facilitate amplicon formation and in some cases increase the rate of amplicon formation. An example is a recombinase. Recombinase can facilitate amplicon formation by allowing repeated invasion/extension. More specifically, recombinase can facilitate invasion of a target nucleic acid by the polymerase and extension of a primer by the polymerase using the target nucleic acid as a template for amplicon formation. This process can be repeated as a chain reaction where amplicons produced from each round of invasion/extension serve as templates in a subsequent round. The process can occur more rapidly than standard PCR since a denaturation cycle (e.g. via heating or chemical denaturation) is not required. As such, recombinase-facilitated amplification can be carried out isothermally. It is generally desirable to include ATP, or other nucleotides (or in some cases non-hydrolyzable analogs thereof) in a recombinase-facilitated amplification reagent to facilitate amplification. A mixture of recombinase and single stranded binding (SSB) protein is particularly useful as SSB can further facilitate amplification. Exemplary formulations for recombinase-facilitated amplification include those sold commercially as TwistAmp kits by TwistDx (Cambridge, UK).

Another example of a component that can be included in an amplification reagent to facilitate amplicon formation and in some cases to increase the rate of amplicon formation is a helicase. Helicase can facilitate amplicon formation by allowing a chain reaction of amplicon formation. The process can occur more rapidly than standard PCR since a denaturation cycle (e.g. via heating or chemical denaturation) is not required. As such, helicase-facilitated amplification can be carried out isothermally. A mixture of helicase and single stranded binding (SSB) protein is particularly useful as SSB can further facilitate amplification. Exemplary formulations for helicase-facilitated amplification include those sold commercially as IsoAmp kits from Biohelix (Beverly, Mass.).

Yet another example of a component that can be included in an amplification reagent to facilitate amplicon formation and in some cases increase the rate of amplicon formation is an origin binding protein.

An advantage of the methods set forth herein is that they provide for rapid and efficient detection of a plurality of target nucleic acid in parallel. Accordingly the present disclosure provides integrated systems capable of preparing and detecting nucleic acids using techniques known in the art such as examples above. Thus, an integrated system of the present disclosure can include fluidic components capable of delivering amplification reagents and/or sequencing reagents to one or more immobilized DNA fragments, the system including components such as pumps, valves, reservoirs, fluidic lines, temperature control, and the like. A flow cell can be configured and/or used in an integrated system for detection of target nucleic acids. As exemplified for flow cells, one or more of the fluidic components of an integrated system can be used for an amplification method and for a detection method. One or more of the fluidic components of an integrated system can be used for an amplification method set forth herein and for the delivery of sequencing reagents in a sequencing method such as those exemplified above. As used herein, the term “flow cell” is intended to mean a vessel having a chamber (i.e., flow channel) where a reaction can be carried out, an inlet for delivering reagent(s) to the chamber, and an outlet for removing reagent(s) from the chamber. In some examples, the chamber enables the detection of a reaction or signal that occurs in the chamber. For example, the chamber can include one or more transparent surfaces allowing for the optical detection of arrays, optically labeled molecules, or the like, in the chamber. As used herein, a “flow channel” or “flow channel region” may be an area defined between two bonded components, which can selectively receive a liquid sample. In some examples, the flow channel may be defined between a patterned support and a lid, and thus may be in fluid communication with one or more depressions defined in the patterned support. In other examples, the flow channel may be defined between a non-patterned support and a lid. Other examples may include dishes, plates, or wells for segregation of reactants, including automated fluidics for exchange of reagents and other components of reactions. For example, multi-well plates may be used, including, for example, 96- or 384-well plates.

Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, Calif.).

Non-limiting examples of suitable primers include P5 and/or P7 primers, which are used on the surface of commercial flow cells sold by Illumina, Inc., for sequencing on HISEQ™, HISEQX™, MISEQ™, MISEQDX™, MINISEQ™, NEXTSEQ™, NEXTSEQDX™, NOVASEQ™, GENOME ANALYZER™, ISEQ™, cBot with imaging (icBot) and other instrument platforms. And portion of a template polynucleotide that includes a nucleotide sequence corresponding to, or complementary to, a first or second primer as disclosed above may have, for example, a sequence corresponding to or complementary to a P5 primer (including a nucleotide sequence of AATGATACGGCGACCACCGAGATCTACAC, SEQ ID NO: 1), a P7 primer (including a nucleotide sequence of CAAGCAGAAGACGGCATACGAGAT, SEQ ID NO: 2), or both, in accordance with such primer sequences as used in the above-mentioned SBS platforms, or others.

A substrate may include, as non-limiting examples, substrates used in any of the aforementioned SBS or other platforms, such as platforms for automated clustering and imaging labelled oligonucleotides hybridized to surface-attached polynucleotides, which may also though need not be a platform equipped for performing sequencing aspects per se of an SBS process. Such a substrate may be a flow cell.

As used herein, the term “depression” refers to a discrete concave feature in a patterned support having a surface opening that is completely surrounded by interstitial region(s) of the patterned support surface. Depressions can have any of a variety of shapes at their opening in a surface including, as examples, round, elliptical, square, polygonal, star shaped (with any number of vertices), etc. The cross-section of a depression taken orthogonally with the surface can be curved, square, polygonal, hyperbolic, conical, angular, etc. As an example, the depression can be a well. Also as used herein, a “functionalized depression” refers to the discrete concave feature where primers are attached, in some examples being attached to the surface of the depression by a polymer (such as a PAZAM or similar polymer).

It is to be understood that the ranges provided herein include the stated range and any value or sub-range within the stated range. As an example, a range from about 100 nm to about 1,000 nm should be interpreted to include not only the explicitly recited limits of from about 100 nm to about 1,000 nm, but also to include individual values, such as about 708 nm, about 945.5 nm, etc., and sub-ranges, such as from about 425 nm to about 825 nm, from about 550 nm to about 940 nm, etc. Furthermore, when “about” and/or “substantially” are/is utilized to describe a value, they are meant to encompass minor variations (up to +/−10%) from the stated value.

### EXAMPLES

The following examples are intended to illustrate particular examples of the present disclosure, but are by no means intended to limit the scope thereof.

### Example 1. An Evaluation of Linearity with Different Polynucleotide Sizes (Human 350 bp, 450 bp and 550 bp and Bacteria 350 bp and 550 bp Libraries) and with Different GC/AT Contents (Bacteria Libraries)

Method: Different ratios of different libraries (population lengths, GC/AT contents, human or bacteria libraries) were used for clustering on HiSeq™ X flow cells. The intensities against the proportional amount of DNA libraries were plotted. Linear fit was applied and R² was calculated with JMP software. Clustering and hybridization of fluorescently labeled oligonucleotide probe for identifier sequence was imaged on an icBot platform. Polynucleotides were tagged with either of two identifier sequences complementary to fluorescently labeled oligonucleotide probes labeled with fluorescent label Alexa 647 (probe 1: /5Alex647N/CT ACA CAT AGA GGC ACA CTC or probe 2: /5Alex647N/CT ACA CGT ACT GAC ACA CTC, available from IDT). Solutions with the following concentrations of polynucleotides from one or another population (or library) were loaded onto 8 flow cell (FC) lanes:

| | Population | Population |
|---|---|---|
| FC | 1 | 2 |
| Lane 1 | 0% | 100% |
| Lane 2 | 20% | 80% |
| Lane 3 | 40% | 60% |
| Lane 4 | 50% | 50% |
| Lane 5 | 60% | 40% |
| Lane 6 | 80% | 20% |
| Lane 7 | 100% | 0% |
| Lane 8 | 50% | 50% |

Gain (40) and imaging exposure time (600 ms for probe 1 and 600 ms-900 ms for probe 2), number of exposures (3), and probe incubation time (6 minutes) were used for imaging surface attached copies after clustering.

In this example, fluorescence intensity is used as a readout to the cluster amplification level. It is important to see if the fluorescence intensity that is detected in this assay correlates to the level of cluster amplification/amount of library input. The good/linear correlation of these two factors establishes the fundamental basis for the assay (and the data analysis method for this assay).

Results

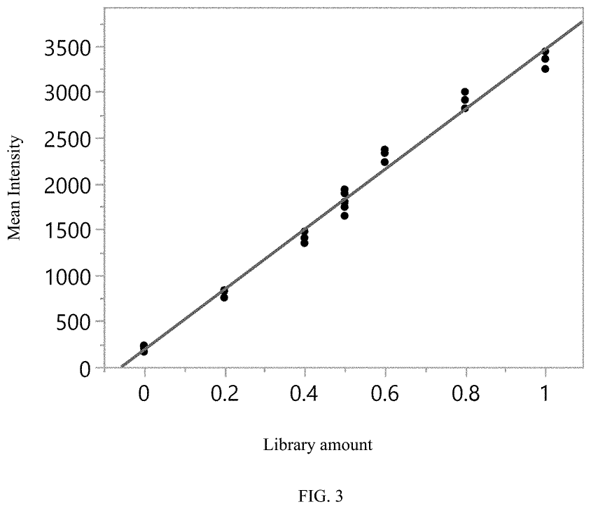

Human 350 bp library: Both probe 1 and probe 2 had similar linearity with R²˜0.99 showing a linear relationship between library input amount (cluster amplification) and signal intensity. Data from probe 2 was shown in FIG. 3. Similarly linear relationships were found using proportions of polynucleotides from GC-rich (e.g., Rhodobacter), GC-poor (e.g., Bacillus cereus), or of longer (550 bp) populations, indicating an translation of concentration of starting material to signal intensity after clustering.

### Example 2: Assay Sensitivity, to Determine how Much Percent DNA Amplification Difference can be Detected

Method: DNA inputs with 10% difference were clustered on HiSeq™ X flow cells. Signal intensities were measured after probe 1 or probe 2 hybridization.

Results

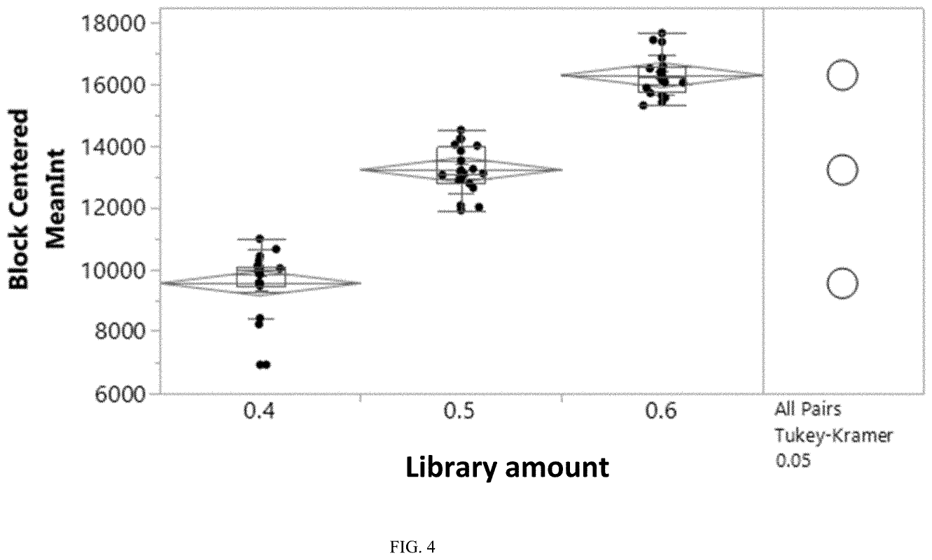

Data from 13 flow cells that were clustered with Rhodobacter 350 bp are summarized in FIG. 4. IMP analysis showed that the DNA library input for Rhodobacter 350 bp library at 40%, 50% and 60% could be separated by this assay with statistical significance (95%). Similar assay sensitivity was found using libraries with different insert size or GC contents, e.g., human 350 bp, 450 bp and 550 bp, Rhodobacter 550 bp, and Bacillus cereus 350 bp and 550 bp libraries.

It should be appreciated that all combinations of the foregoing concepts and additional concepts discussed in greater detail herein (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein and may be used to achieve the benefits and advantages described herein.

1. A method, comprising
making copies of two or more populations of polynucleotides comprising identifier sequences, wherein the copies are attached to a substrate,
hybridizing oligonucleotides to the identifier sequences, and
comparing an amount of oligonucleotides hybridized to the copies of the two or more populations of polynucleotides, wherein
at least one feature differs between the two or more populations of polynucleotides or between the making of the copies of the two or more populations of polynucleotides attached to the substrate.

2. The method of claim 1, wherein the at least one feature is selected from a length, a guanine-cytosine content, and a preparation method.

3. The method of claim 1, wherein the at least one feature comprises a guanine-cytosine content.

4. The method of claim 1, wherein the at least one feature comprises a length.

5. The method of claim 1, wherein the at least one feature comprises a preparation method.

6. The method of claim 1, wherein at least one feature differs between the making of the copies of the two or more populations of polynucleotides attached to the substrate.

7. The method of claim 1, wherein the oligonucleotides comprise a fluorophore.

8. The method of claim 2, further comprising detecting a difference between amounts of oligonucleotides hybridized to the copies of the two or more populations of polynucleotides attached to the substrate, wherein the difference is at least about 10%.

9. The method of claim 8, wherein the difference is at least about 20%.

10. The method of claim 8, wherein the difference is at least about 30%.

11. The method of claim 1, wherein:
the at least one feature comprises a combination and the combination comprises two or more of a guanine-cytosine content, a length, a preparation method, and the making of the copies of the two or more populations of polynucleotides attached to the substrate,
the two or more populations of polynucleotides comprise three or more populations of polynucleotides, and
the combination of each of the three or more populations of polynucleotides differs from the combination of another population of polynucleotides.

12. The method of claim 11, further comprising detecting a difference between amounts of oligonucleotides hybridized to the copies of two or more of the three or more populations of polynucleotides attached to the substrate, wherein the difference is at least about 10%.

13. The method of claim 12, wherein the difference is at least about 20%.

14. A method, comprising
making copies of two or more populations of polynucleotides comprising identifier sequences, wherein the copies are attached to a substrate,
hybridizing oligonucleotides comprising a fluorophore to the identifier sequences, and
detecting an amount of oligonucleotides hybridized to the copies of the two or more populations of polynucleotides, wherein
at least one feature differs between the two or more populations of polynucleotides or between the making of the copies of the two or more populations of polynucleotides attached to the substrate, and
the at least one feature is selected from a length, a guanine-cytosine content, a preparation method, and the making of the copies of the two or more populations of polynucleotides attached to the substrate.

15. The method of claim 14, wherein the at least one feature comprises a guanine-cytosine content.

16. The method of claim 14, wherein the at least one feature comprises a length.

17. The method of claim 14, wherein the at least one feature comprises a preparation method.

18. The method of claim 14, wherein at least one feature differs between the making of the copies of the two or more populations of polynucleotides attached to the substrate.

19. The method of claim 14, further comprising detecting a difference between an amount of oligonucleotides hybridized to copies of the two or more populations of polynucleotides, wherein the difference is at least about 10%.

20. The method of claim 19, wherein the difference is at least about 20%.
